(19) United States
(12) Patent Application Publication
(10) Pub. No.: US 2009/0258816 A1
(43) Pub. Date: Oct. 15, 2009

(54) USE OF GPCR AGONISTS TO DELAY PROGRESSION OF DIABETES

(76) Inventors: Matthew Colin Thor Fyee, Oxford (GB); Peter Widdowson, Oxford (GB)

Correspondence Address: OSI PHARMACEUTICALS, INC., 41 PINELAWN ROAD, MELVILLE, NY, 11747, US

(21) Appl. No.: 12/303,028

(22) PCT Filed: Jun. 1, 2007
(86) PCT No.: PCT/GB07/50313

§ 371 Date: Dec. 1, 2008

(30) Foreign Application Priority Data
Jun. 1, 2006 (GB) 0610746.0

(51) Int. Cl. A61K 38/02; A61P 3/10

(52) U.S. Cl. 514/2

(57) ABSTRACT

The present invention is directed to the use of G-protein coupled receptor agonists for the treatment of beta-cell degeneration.

### BACKGROUND OF THE INVENTION

The present invention is directed to the use of G-protein coupled receptor (GPCR) agonists. In particular, the present invention is directed to the use of agonists of GPR119 for the treatment of beta-cell degeneration and to delay the progression of the pre-diabetic state or type 2 diabetes.

Obesity is characterized by an excessive adipose tissue mass relative to body size. Clinically, body fat mass is estimated by the body mass index (BMI; weight(kg)/height(m)²), or waist circumference. Individuals are considered obese when the BMI is greater than 30 and there are established medical consequences of being overweight. It has been an accepted medical view for some time that an increased body weight, especially as a result of abdominal body fat, is associated with an increased risk of diabetes.

Diabetes mellitus is a chronic metabolic disorder characterized by the presence of hyperglycaemia (raised blood glucose concentrations). The prevalence of type 2 diabetes or non-insulin-dependent diabetes mellitus (NIDDM), is high and is growing at an alarming rate. The global burden of diabetes mellitus is expected to reach 300 million by the year 2025, with more than 90% of these individuals having type 2 diabetes.

Pre-diabetes, often referred to as impaired glucose tolerance or impaired fasting glycemia (see Definition and Classification of Diabetes Mellitus and its Complications. Report of a WHO Consultation. Geneva, 1999, WHO/NCD/NCS 99.2), is a condition where blood glucose levels are above normal but not high enough to be diagnosed as type 2 diabetes.

The predominant pathophysiological defects leading to hyperglycaemia in type 2 diabetes are impaired insulin action (insulin resistance) and impaired insulin secretion (beta-cell dysfunction). Treating hyperglycaemia is therapeutically important in diabetes mellitus in order to prevent symptoms caused by the raised blood glucose concentrations, such as polyuria (excessive urination) and polydipsia (excessive thirst), and to reduce the risk of diabetic complications. The chronic hyperglycaemia of diabetes mellitus is associated with significant, often devastating long-term complications in the eyes, kidneys, nerves and blood vessels. The largest study of pharmacotherapy in type 2 diabetes, The United Kingdom Prospective Diabetes Study (UKPDS), [Diabetes 44:1249-1258, 1995] also demonstrated that an inexorable decline in beta-cell function occurs with time in type 2 diabetes. Beta-cell degeneration leads, in the majority of patients, to worsening of glycaemic control with time, requiring addition of more and more therapies as the disease progresses leading eventually to the patient becoming dependent on the administration of insulin. This decline in beta-cell function will generally have begun in a patient during the pre-diabetic state and much earlier than the diagnosis of the patient as having type 2 diabetes. It is estimated that a patient may already have lost 40% of their β-cell function at the point of diagnosis. However, it is only at the point that the patient is diagnosed as having elevated blood glucose levels that they will be prescribed a blood glucose lowering agent.

There are a number of oral agents currently available to treat type 2 diabetes. Commonly prescribed agents are metformin and the sulphonylureas. Metformin acts by decreasing glucose output from the liver, it is associated with gastrointestinal side-effects in many patients and has no impact on the decline in beta-cell function with time. The sulphonylureas act by increasing insulin secretion, are associated with the side effects of weight gain and hypoglycaemia (low blood glucose concentrations) and, like metformin, have no impact on the decline in beta-cell function with time (see UKPDS).

There is a continuing need for agents which are capable of treating beta-cell degeneration and of delaying the progression of the pre-diabetic state or type 2 diabetes.

GPR119 is a GPCR identified as SNORF25 in WO00/50562 which discloses both the human and rat receptors, U.S. Pat. No. 6,468,756 also discloses the mouse receptor (accession numbers: AAN95194 (human), AAN95195 (rat) and ANN95196 (mouse)). In humans, GPR119 is expressed in the pancreas, small intestine, colon and adipose tissue which are target sites for the regulation of insulin, incretins and food intake. The expression profile of the human GPR119 receptor indicates its potential utility as a target for the treatment of obesity and diabetes.

International patent applications WO20041065380, WO2004/076413, WO2005/007647, WO2005/007658, WO2005/121121, WO2005/061489, WO2006/067531, WO2006/067532, WO2006/070208, WO2006/083491, WO2007/003960, WO2007/003961, WO2007/003962, WO2007/003964 and WO2007/035355 disclose small molecule GPR119 agonists.

### SUMMARY OF THE INVENTION

The present invention is directed to the use of agonists of GPR119 for the treatment of beta-cell degeneration and to delay the progression of the pre-diabetic state or type 2 diabetes.

### DETAILED DESCRIPTION OF THE INVENTION

The invention provides a method for the treatment beta-cell degeneration comprising administering to a patient in need thereof an effective amount of a GPR119 agonist.

Beta-call degeneration includes the worsening of beta-cell function (beta-cell dysfunction) and/or the loss of beta-cells through apoptosis or necrosis.

The GPR119 agonists may treat beta-cell degeneration by inhibiting or decreasing the worsening of beta-cell function.

The GPR119 agonists may also treat beta-cell degeneration by increasing the number or size of beta-cells. The number and/or size of beta-cells may be increased by causing pancreatic cells to proliferate to functionally active cells of the islets of Langerhans and/or by causing transformation of insensitive or impaired pancreatic cells into functionally active cells of the islets of Langerhans.

Thus according to a further aspect the invention provides a method for increasing the number or size of beta-cells comprising administering to a patient in need thereof an effective amount of a GPR119 agonist.

As the GPR119 agonists treat beta-cell degeneration they are useful for delaying the progression of the pre-diabetic state to type 2 diabetes and also for delaying the progression of type 2 diabetes e.g. to the point where the patient becomes dependent on the administration of insulin to achieve adequate glycemic control.

Therefore the invention also provides a method for delaying the progression of the pre-diabetic state to type 2 diabetes comprising administering to a patient in need thereof an effective amount of a GPR119 agonist.

The invention also provides a method for delaying the progression of type 2 diabetes comprising administering to a patient in need thereof an effective amount of a GPR119 agonist.

The invention also provides a GPR119 agonist for use in the treatment of a condition as defined above.

The invention also provides the use of a GPR119 agonist in the manufacture of a medicament for the treatment of a condition as defined above.

In the methods of the invention the term “treatment” includes both therapeutic and prophylactic treatment.

The patient to be treated according to the invention is preferably a human.

The GPR119 agonists for use in the method of the invention include peptides, polypeptides, proteins, enzymes, antibodies as well as non-peptides, e.g. small molecules. The GPR119 agonist is preferably an orally acting small molecule, for example an organic small molecule having a molecular weight of preferably less than 800, more preferably less than 600, especially less than 500. The GPR119 agonist may be a compound described in WO2004/065380, WO2004/076413, WO2005/007647, WO2005/007658, WO2005/121121, WO20051061489, WO2006/067531, WO2006/067532, WO2006/070208. WO2006/08349 1, WO2007/003960, WO2007/003961, WO2007/003962, WO20071003964 or WO2007/035355.

For use in the methods of the invention the GPR119 agonist will generally be administered in the form of a pharmaceutical composition.

The invention also provides a pharmaceutical composition for the treatment of beta-cell degeneration comprising a pharmaceutically acceptable carrier and a therapeutically effective amount of a GPR119 agonist.

The pharmaceutical compositions may optionally comprise other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the GPR119 agonist can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous).

Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the GPR119 agonist may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The GPR119 agonist can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, using a GPR119 agonist via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a GPR119 agonist may also be prepared in powder or liquid concentrate form.

Generally, dosage levels on the order of 0.01 mg/kg to about 150 mg/kg of body weight per day are useful in the treatment of the above-indicated conditions, or alternatively about 0.5 mg to about 7 g per patient per day. For example, obesity may be effectively treated by the administration of from about 0.01 to 50 mg of the GPR119 agonist per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The GPR119 agonist may be administered with other active compounds for the treatment of obesity and/or diabetes, for example insulin and insulin analogs, gastric lipase inhibitors, pancreatic lipase inhibitors, sulfonyl ureas and analogs, biguanides e.g. metformin, α2 agonists, glitazones, PPAR-γ agonists, mixed PPAR-α/γ agonists, RXR agonists, fatty acid oxidation inhibitors, α-glucosidase inhibitors, glucokinase activators, dipeptidyl peptidase IV inhibitors, GLP-1 agonists e.g. GLP-1 analogues and mimetics, βagonists, phosphodiesterase inhibitors, lipid lowering agents, glycogen phosphorylase inhibitors, antiobesity agents e.g. pancreatic lipase inhibitors, MCH-1 antagonists and CB-1 antagonists (or inverse agonists), amylin antagonists, lipoxygenase inhibitors, somostatin analogs, glucokinase activators, glucagon antagonists, insulin signalling agonists, PTP1B inhibitors, gluconeogenesis inhibitors, antilypolitic agents, ASK inhibitors, galanin receptor agonists, anorectic agents, CCK receptor agonists, leptin, serotonergic/dopaminergic antiobesity drugs, reuptake inhibitors e.g. sibutramine, CRF antagonists, CRF binding proteins, thyromimetic compounds, aldose reductase inhibitors, glucocorticoid receptor antagonists, NHE-1 inhibitors or sorbitol dehydrogenase inhibitors.

The GPR119 agonist and the other agent(s) may be co-administered or administered sequentially or separately.

Co-administration includes administration of a formulation which includes both the GPR119 agonist and the other agent(s), or the simultaneous or separate administration of different formulations of each agent. Where the pharmacological profiles of the GPR119 agonist and the other agent(s) allow it, coadministration of the two agents may be preferred.

All publications, including, but not limited to, patents and patent application cited in this specification, are herein incorporated by reference as if each individual publication were specifically and individually indicated to be incorporated by reference herein as fully set forth.

The invention will now be described by reference to the following examples which are for illustrative purposes and are not to be construed as a limitation of the scope of the present invention.

### BRIEF DESCRIPTION OF THE FIGURES

FIG. 1 shows the effect of GPR119 agonists in preventing diabetes in young db/db mice.

### EXAMPLES

The activity of compounds as GPR119 agonists may be tested in the following assay systems:
1) Yeast Reporter Assay

The yeast cell-based reporter assays have previously been described in the literature (e.g. see Miret J. J. et al, 2002, J. Biol. Chem., 277:6881-6887; Campbell R. M. et al, 1999, Bioorg. Med. Chem. Lett., 9:2413-2418; King K. et al, 1990, Science, 250:121-123); WO 99/14344; WO 00/12704; and U.S. Pat. No. 6,100,042). Briefly, yeast cells have been engineered such that the endogenous yeast G-alpha (GPA1) has been deleted and replaced with G-protein chimeras constructed using multiple techniques. Additionally, the endogenous yeast alpha-cell GPCR, Stc3 has been deleted to allow for a homologous expression of a mammalian GPCR of choice. In the yeast, elements of the pheromone signaling transduction pathway, which are conserved in eukaryotic cells (for example, the mitogen-activated protein kinase pathway), drive the expression of Fus1. By placing galactosidase (LacZ) under the control of the Fus1 promoter (Fus1p), a system has been developed whereby receptor activation leads to an enzymatic read-out.

Yeast cells were transformed by an adaptation of the lithium acetate method described by Agatep et al, (Agatep, R. et al, 1998, Transformation of Saccharomyces cerevisiae by the lithium acetate/single-stranded carrier DNA/polyethylene glycol (LiAc/ss-DNA/PEG) protocol. Technical Tips Online, Trends Journals, Elsevier). Briefly, yeast cells were grown overnight on yeast tryptone plates (YT). Carrier single-stranded DNA (10 μg), 2 ∞g of each of two Fus1p-LacZ reporter plasmids (one with URA selection marker and one with TRP), 2 μg of GPR116 (human or mouse receptor) in yeast expression vector (2 μg origin of replication) and a lithium acetate/polyethylene glycol/TE buffer was pipetted into an Eppendorf tube. The yeast expression plasmid containing the receptor/no receptor control has a LEU marker. Yeast cells were inoculated into this mixture and the reaction proceeds at 30° C. for 60 min. The yeast cells were then heat-shocked at 42° C. for 15 min. The cells were then washed and spread on selection plates. The selection plates are synthetic defined yeast media minus LEU, URA and TRP (SD-LUT). After incubating at 30° C. for 2-3 days, colonies that grow on the selection plates were then tested in the LacZ assay.

In order to perform fluorimetric enzyme assays for β-galactosidase. yeast cells carrying the human or mouse GPR119 receptor were grown overnight in liquid SD-LUT medium to an unsaturated concentration (i.e. the cells were still dividing and had not yet reached stationary phase). They were diluted in fresh medium to an optimal assay concentration and 90 μl of yeast cells are added to 96-well black polystyrene plates (Costar). Compounds, dissolved in DMSO and diluted in a 10% DMSO solution to 10× concentration, were added to the plates and the plates placed at 30° C. for 4 h. After 4 h, the substrate for the β-galactosidase was added to each well. In these experiments, Fluorescein di (β-D-galactopyranoside) was used (FDG), a substrate for the enzyme that releases fluorescein. allowing a fluorimetric read-out. 20 μl per well of 500 μM FDG/2.5% Triton X100 was added (the detergent was necessary to render the cells permeable). After incubation of the cells with the substrate for 60 min, 20 μl per well of 1 M sodium carbonate was added to terminate the reaction and enhance the fluorescent signal. The plates were then read in a fluorimeter at 485/535 nm.

GPR119 agonists will generally give an increase in fluorescent signal of at least ˜1.5-fold that of the background signal (i.e. the signal obtained in the presence of 1% DMSO without compound).
2) cAMP Assay

A stable cell line expressing recombinant human GPR119 was established and this cell line was used to investigate the effect of compounds on intracellular levels of cyclic AMP (cAMP). The cells monolayers were washed with phosphate buffered saline and stimulated at 37° C. for 30 min with various concentrations of compound in stimulation buffer plus 1% DMSO. Cells were then lysed and cAMP content determined using the Perkin Elmer AlphaScreen™ (Amplified Luminescent Proximity Homogeneous Assay) cAMP kit. Buffers and assay conditions were as described in the manufacturer's protocol.

GPR119 agonists will generally show a concentration-dependant increase in intracellular cAMP level and e.g. have an EC₅₀ of <10 μM.

The effects of a GPR119 agonist in preventing diabetes in young db/db mice may be demonstrated as follows.

GPR119 agonists were evaluated in prediabetic 6 week old db/db mice. Mice were kept in a 12 hour light/dark cycle with lights on at 7.00 h. Mice were dosed daily at 9.00 h with vehicle (25% ag. Gelucire 44/14, p.o.) or GPR119 agonist (100 mg/kg p.o. in 25% ag. Gelucire 44/14) for 21 days. On days 0, 7 and 21 oral glucose tolerance tests (OGTT) were conducted with GIc load (1.5 g kg⁻¹ p.o.). On these days, compound was dosed after the OGTT, at 11.00 h. During the OGTTs, blood samples (20 μL) were then taken 25, 50, 80, and 120 min after Glc administration. The 20 μL blood samples for measurement of GIc levels were taken from the cut tip of the tail into disposable micro-pipettes (Dade Diagnostics Inc., Puerto Rico) and the sample added to 480 μL of haemolysis reagent. Duplicate 20 μL aliquots of the diluted haemolysed blood were then added to 180 μL of Trinders glucose reagent (Sigma enzymatic (Trinder) colorimetric method) in a 96-well assay plate. After mixing, the samples were left at rt for 30 min before being read against Glc standards (Sigma glucose/urea nitrogen combined standard set). 30 min after Glc administration a blood sample was taken for insulin testing. Fed blood glucose levels were measured on day 22. Insulin concentrations, using 5 μL of plasma, were measured using a 96-well ELISA kit (Crystal Chem. Inc. #INSKR020 96 assays) according to instructions provided by the manufacturer.

Results of plasma glucose and insulin levels expressed as the mean ±SEM (mM and pM, respectively). The statistical analysis consisted of a one-way analysis of variance coupled with t-tests for each time point A difference is considered significant for p<0.05. For the OGTT studies, AUC (0-120 min) was also calculated and delta blood values calculated.

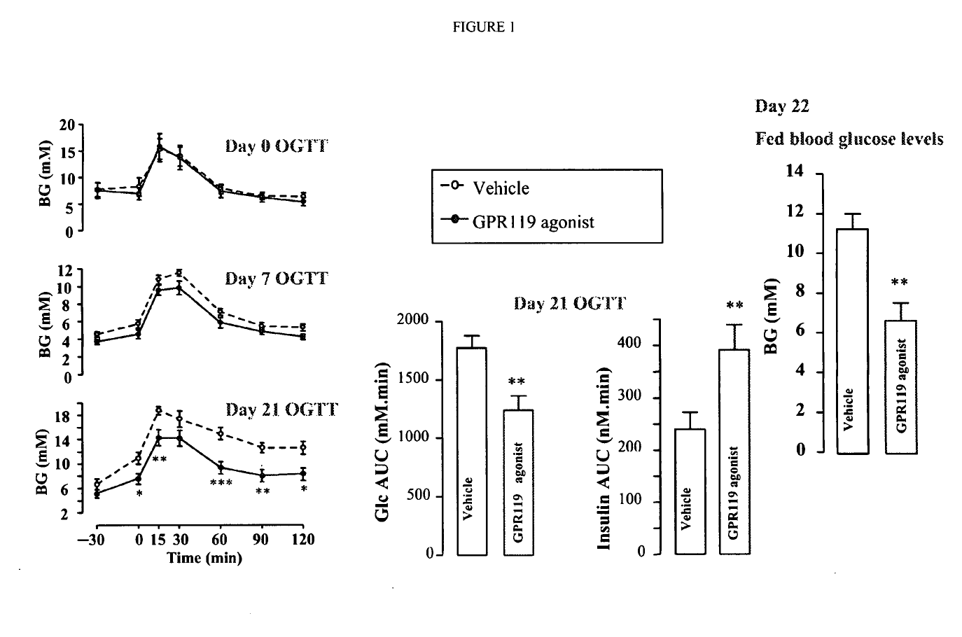

Over the 21 day treatment period, the mice dosed with GPR119 agonist showed an oral glucose tolerance profile that was equivalent to pre-diabetic db/db mice, whereas control mice that were dosed with vehicle, showed a raised fasting glucose concentration and a degree of glucose intolerance. These data are consistent with the development of diabetes in vehicle-treated animals, whereas treatment with a GPR119 agonist over the critical period during which db/db mice become diabetic, prevents or delays the diabetic condition (FIG. 1). Moreover, the ability of GPR119 agonist-treated db/db mice to significantly enhance insulin secretion, relative to vehicle-treated diabetic db/db mice after 21 days therapy show that sustained activation of GPR119 receptors can enhance pancreatic beta-cell function in response to glucose challenge and is indicative of an attenuation in the decline of beta-cell function.

The effects of a GPR119 agonist on diabetes progression in ZDF rats may be demonstrated as follows.

GPR119 agonists were evaluated in prediabetic 6 week old ZDF rats. Rats were kept in a 12 hour light/dark cycle with lights on at 6.00 h. Rats were dosed daily at 8.15 h with vehicle (20% aqueous hydroxypropyl-beta-cyclodextrin, u.i.d. oral.) or GPR119 agonist (10 or 30 mg/kg u.i.d. oral, in 20% aqueous hydroxypropyl-beta-cyclodextrin) for 56 days. On days 1, 29 and 56 oral glucose tolerance tests (OGTT) were conducted with Glc load (2 g kg⁻¹ p.o.) 45 min after dosing of vehicle of GPR119 agonist. During the OGTTs, blood samples (20 μL) were taken 0, 15, 30, 45, 60, 90, 120, 150 and 180 min after Glc administration. The 20 μL blood samples for measurement of Glc levels were taken from the cut tip of the tail into disposable micro-pipettes and placed in standard tubes filled with 1 ml solution for haemolysis (blood glucose measurement) and in sample tubes for plasma insulin. Blood glucose levels were measured using the glucose oxidase procedure (Super G Glukose Analyser, Dr Müller Gerätebau, Freital. Germany) and plasma insulin concentrations asscd by ELISA (Mercodia AB, Uppsala, Sweden).

At the start of the study, the rats were six weeks old, and, as a result, were not diabetic. The ZDF rats treated with vehicle rapidly became diabetic, as illustrated both by a sharp rise in fed glucose levels and by markedly increased water intake, a result of the polydipsia that accompanies the polyuria associated with glucose loss in the urine. Three weeks into the study, the fed blood glucose concentrations in the control ZDF rats, dosed with vehicle, had increase 2-3-fold. In contrast, the rise in glucose levels in the rats treated with the GPR119 agonist was more gradual, leading to these animals exhibiting significantly lower fed blood glucose concentrations than those in their vehicle-treated counterparts. For instance, in the 3-6 week period of the study, fed blood glucose concentrations were 6-7 mM lower in the rats given 30 mg/kg/d GPR119 agonist than in those given vehicle. The GPR119 agonist reduced polydipsia, another parameter correlated with the progression of diabetes. The GPR119 agonist also significantly attenuated long-term glucose exposure, as indicated by a smaller rise in Hb_{Alc} levels, compared to vehicle-treated animals. In the OGTTs the GPR119 agonist displayed strong antihyperglycaemic effects throughout the eight weeks of dosing. In contrast to the vehicle treated animals the glucose tolerance of the GPR119 treated animals, as revealed by significantly decreased reactive glucose AUCs, remained similar throughout the study

Thus, sustained activation of GPR119 appeared to attenuate disease progression during the period of diabetes development in ZDF rats.

The effects of GPR119 agonists on beta-cell function may also be measured in animal models as described in “Dipeptidyl peptidase IV inhibitor treatment stimulates β-cells survival and islet cell neogenesis in streptozotocin-induced diabetic rats” by Popisilik et al, Diabetes, 52: 741-750, 2003.

What is claimed is:

1. A method for the treatment of beta-cell degeneration comprising administering to a patient in need thereof an effective amount of a GPR119 agonist.

2. The method according to claim 1 wherein the GPR119 agonist treats beta-cell degeneration by inhibiting or decreasing the worsening of beta-cell function and/or the loss of beta-cells through apoptosis or necrosis.

3. The method according to claim 1 wherein the GPR119 agonist increases the number or size of beta-cells.

4. The method according to claim 3 wherein the GPR119 agonist causes pancreatic cells to proliferate to functionally active cells of the islets of Langerhans and/or causes transformation of insensitive or impaired pancreatic cells into functionally active cells of the islets of Langerhans.

5. A method for delaying the progression of the pre-diabetic state to type 2 diabetes comprising administering to a patient in need thereof an effective amount of a GPR119 agonist.

6. A method for delaying the progression of type 2 diabetes comprising administering to a patient in need thereof an effective amount of a GPR119 agonist.

7. The method according to claim 1 wherein the patient to be treated is a human.

8. The method according to claim 1 wherein the GPR119 agonist is an orally acting small molecule.

9. The method according to claim 5 wherein the patient to be treated is a human.

10. The method according to claim 6 wherein the patient to be treated is a human.

11. The method according to claim 5 wherein the GPR119 agonist is an orally acting small molecule.

12. The method according to claim 6 wherein the GPR119 agonist is an orally acting small molecule.
